(19) United States
(12) Patent Application Publication
(10) Pub. No.: US 2025/0083088 A1
(43) Pub. Date: Mar. 13, 2025

(54) METHODS OF BUTANE DELIVERY

(71) Applicant: Texon Midstream, LLC, Houston, TX (US)

(72) Inventor: John Colin Robbins, Houston, TX (US)

(21) Appl. No.: 18/827,752

(22) Filed: Sep. 8, 2024

Related U.S. Application Data
(60) Provisional application No. 63/537,718, filed on Sep. 11, 2023.

(51) Int. Cl. B01D 53/00; C07C 7/09; F17C 7/02; F25J 3/06

(52) U.S. Cl. CPC B01D 53/002; C07C 7/09; F17C 7/02; F25J 3/064; F25J 3/0645; F25J 3/065; B01D 2256/10; B01D 2257/7022; B01D 2259/4525; B01D 2259/455; F17C 2221/035; F25J 2210/42; F25J 2215/62; F25J 2215/64; F25J 2215/66

(57) ABSTRACT

Methods of butane delivery, methods for reducing VOC emissions resulting from such delivery, and methods for debutanizing nitrogen gas used to expel butane from butane storage tanks.

### FIELD OF THE INVENTION

The present invention relates to methods of butane delivery, methods for reducing VOC emissions resulting from such delivery, and methods for debutanizing nitrogen gas used to expel butane from butane storage tanks.

### BACKGROUND OF THE INVENTION

Volatile Organic Compounds (VOCs) are organic compounds that have an initial boiling point less than or equal to 250° C. (482° F.) when measured at a standard atmospheric pressure of 101.3 kPa. VOCs are a mixture of light end hydrocarbons (such as methane, ethane, propane or butane) that evaporate within the range of normal atmospheric conditions. In storage tanks including stationary storage tanks and cargo storage tanks such as rail cars and barge holding tanks, VOCs are commonly generated from evaporation of liquid hydrocarbons inside the tanks during and after loading. VOC generation is particularly acute when inert gases such as nitrogen are used as pressurizing agents to expel liquid hydrocarbons from the tanks.

Condensation is one technology used to reduce VOC emission rates. In the United States, its use has been driven in part by the Clean Air Act Amendments of 1990, which establish acceptable rates for VOC emissions. Other methods of controlling VOC emissions are described in U.S. Pat. No. 5,294,296 to The Dow Chemical Company.

Additional methods are needed to control emissions of VOCs, particularly from tanks used to transport or store light hydrocarbons such as butane. Ideally, the methods can be used to remove VOCs from the inert gases used to pressurize hydrocarbon tanks when expelling the liquid hydrocarbons from the tanks.

### SUMMARY OF THE INVENTION

Versatile methods and systems have been developed for reducing VOC emissions that arise from the delivery of light hydrocarbons such as butane from storage tanks. The methods and systems are particularly useful when an inert gas such as nitrogen is used as a pressurizing agent to expel the liquid hydrocarbon from the tank, the liquid hydrocarbon volatilizes into the inert gas, and it becomes necessary to remove the hydrocarbon from the inert gas before the gas can be vented to the atmosphere.

Thus, in a first principal embodiment the invention provides a method of delivering butane comprising: (a) providing a pressurized butane storage tank comprising a liquid consisting essentially of butane and a gaseous headspace, wherein the tank comprises a liquid inlet port and a liquid outlet port in fluid communication with the liquid and a gas inlet port and a gas outlet port in fluid communication with the gaseous headspace; (b) injecting nitrogen gas into the tank through the gas inlet port at a pressure sufficient to expel liquid butane through the liquid outlet port; (c) retaining the gaseous nitrogen in the tank in the headspace for a time and at a temperature sufficient to form a gaseous mixture of butane and; (d) releasing the gaseous nitrogen from the tank at a first pressure optionally greater than 30 psig; (e) compressing the nitrogen to a second pressure optionally greater than 300 psig and a temperature greater than the condensation point of butane at the second pressure; (f) condensing butane from the nitrogen by reducing the temperature of the nitrogen below the condensation point of butane at the second pressure while maintaining the nitrogen in a gaseous state; and (g) venting the nitrogen to the atmosphere.

In a second principal embodiment the invention provides a method of removing butane from a gaseous nitrogen headspace in a pressurized butane storage tank comprising: (a) releasing the gaseous nitrogen from the tank at a first pressure optionally greater than 30 psig; (b) compressing the nitrogen to a second pressure optionally greater than 300 psig and a temperature greater than the condensation point of butane at the second pressure; (c) condensing butane from the nitrogen by reducing the temperature of the nitrogen below the condensation point of butane at the second pressure while maintaining the nitrogen in a gaseous state; and (d) venting the nitrogen to the atmosphere.

A third principal embodiment relates to mobile systems capable of performing the methods of the current invention. Thus, in a third principal embodiment the invention provides a mobile system for removing butane from a gaseous nitrogen headspace in a pressurized butane storage tank comprising: (a) a mounting platform and a plurality of wheels rotatably secured thereto; (b) means for releasing the gaseous nitrogen at pressures optionally greater than 30 psig from the pressurized butane storage tank, wherein the gaseous nitrogen comprises a partial pressure of gaseous butane; (c) a compressor in fluid communication with the means for releasing, capable of increasing the pressure of the gaseous nitrogen to optionally greater than 300 psig and a temperature greater than the condensation point of butane at the second pressure; (d) a condenser in fluid communication with the compressor, capable of condensing butane from the nitrogen by reducing the temperature of the nitrogen below the condensation point of butane while maintaining the nitrogen in a gaseous state; (e) a high-pressure liquid separator capable of separating the condensed butane from the nitrogen while maintaining the condensed butane in a liquid state; and (f) means for venting the nitrogen to the atmosphere.

Additional advantages of the invention are set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

A better understanding of the present invention can be obtained when the following detailed description of the disclosed embodiments is considered in conjunction with the following drawings, in which:

FIG. 1 is a plan layout of a system for practicing the methods of the current invention on a stationary butane storage tank.

### DETAILED DESCRIPTION

### Definitions and Use of Terms

As used in the specification and claims, the singular forms “a,” “an,” and “the” include plural references unless the context clearly dictates otherwise. The word “or” and like terms as used herein means any one member of a particular list and also includes any combination of members of that list.

The invention is defined in terms of principal embodiments and subembodiments. When an embodiment or subembodiment other than the principal embodiment is discussed herein, it will be understood that the embodiment or subembodiment can be applied to further limit any of the principal embodiments. It will also be understood that the elements and subembodiments can be combined to create other distinct subembodiments encompassed by the present invention.

When an element of a process or thing is defined by reference to one or more examples, components, properties or characteristics, it will be understood that any one or combination of those components, properties or characteristics can also be used to define the subject matter at issue. This might occur, for example, when specific examples of an element are recited in a claim (as in a Markush grouping), or an element is defined by a plurality of characteristics. Thus, for example, if a claimed system comprises element A defined by elements A1, A2 and A3, in combination with element B defined by elements B1, B2 and B3, the invention will also be understood to cover a system defined by element A without element B, a system in which element A is defined by elements A1 and A2 in combination with element B defined by elements B2 and B3, etc.

“Diluent” means hydrocarbon added to crude, heavy naphtha, bitumen or other dense petrochemical material to reduce the viscosity or density of such material. A common source of diluent is natural gas condensate obtained during the extraction of natural gas. Other diluent sources include but are not limited to: light conventional produced hydrocarbon oils, refinery naphtha (i.e. straight run hydrocarbons from the refinery process, especially light naphtha) and synthetic crude oils. “Diluent” refers to hydrocarbons derived from a single source as well as pooled diluent streams. A diluent, by definition, preferably contains substantially virgin (uncracked) hydrocarbons, although trace amounts of cracked hydrocarbons (<5, 2 or 1%) are acceptable.

“Liquid butane” has its usual meaning in liquid fuel contexts; it refers to a composition consisting substantially of C₄H₁₀ hydrocarbons, and as used herein includes both n-butane and isobutane when the isomer is not specified. The term includes commercially available liquid butane in the presence of like-fraction hydrocarbons. In various embodiments, the term “liquid butane” requires the hydrocarbon content to be at least 80%, 90%, 95%, or 98% C₄H₁₀.

When the “partial pressure” of butane in a gas is cited, it will be understood to refer only to the pressure contributed by the butane, including any n-butane and isobutane in the gaseous butane, thus excluding the partial pressure contributed by any other VOCs.

“Finished Gasoline” and “Finished Motor Gasoline” are used synonymously and refer to gasoline that is suitable for burning in spark-ignition vehicles without further modifications.

Finished gasoline will typically satisfy ASTM Specification D 4814 or Federal Specification VV-G-1690C, and is characterized as having a boiling range of 122 to 158 degrees Fahrenheit at the 10 percent recovery point to 365 to 374 degrees Fahrenheit at the 90 percent recovery point. “Conventional Gasoline” means finished motor gasoline not included in the oxygenated or reformulated gasoline categories.

“Fluid communication” refers to the linkage of a pipe to a source of a fluid at the same facility. Optionally the linkage may be through a channel that can be closed or whose flow may be modulated as by a valve. It will be understood that, when a gas or liquid is in fluid communication with an inlet port or an outlet port, that the gas or liquid need not be in physical contact with the port, and that it remains in fluid communication with the port even when separated by a liquid phase (when referring to fluid communication with the gas) or a gaseous phase (when referring to fluid communication with the liquid).

“Gasoline” refers to a refined mixture of relatively volatile hydrocarbons with or without small quantities of additives, blended to form a fuel suitable for use in spark-ignition engines. The term includes finished gasoline and gasoline/ethanol mixtures, as well as fuels that are intended to be mixed with oxygenates such as ethanol and MTBE. Gasoline thus includes conventional gasoline; oxygenated gasoline such as gasohol; reformulated gasoline; reformulated blendstock for oxygenate blending; and conventional blendstock for oxygenate blending.

“Tank farm” means any facility that contains a number of large storage tanks for petroleum products, serviced by bulk transport facilities such as ships or pipelines originating off-site for delivering petroleum products, and often including loading racks from which tanker trucks can be filled. The methods and systems of the current invention occur at tank farms downstream of a petroleum refinery.

Percentages refer to weight percentages when referring to liquids and to partial pressures (i.e. molar percentages) when referring to vapors or gases. The term “nitrogen gas, when referring to nitrogen gas having one or more VOCs mixed therein, includes the partial pressure contributed by the VOCs. In like manner, “liquid butane,” when referring to a pool or volume of butane, includes the entire pool of liquid even when the pool includes hydrocarbons other than butane. The terms “vapor” and “gas” are used synonymously.

When pressures are expressed herein, it will be understood that they refer to psig unless stated otherwise.

### Discussion of Principal Embodiments

In a first principal embodiment the invention provides a method of delivering butane comprising: (a) providing a pressurized butane storage tank comprising a liquid consisting essentially of butane and a gaseous headspace, wherein the tank comprises a liquid inlet port and a liquid outlet port in fluid communication with the liquid and a gas inlet port and a gas outlet port in fluid communication with the gaseous headspace; (b) injecting nitrogen gas into the tank through the gas inlet port at a pressure sufficient to expel liquid butane through the liquid outlet port; (c) retaining the gaseous nitrogen in the tank in the headspace for a time and at a temperature sufficient to form a gaseous mixture of butane and nitrogen; (d) releasing the gaseous nitrogen from the tank at a first pressure optionally greater than 30 psig; (c) compressing the nitrogen to a second pressure optionally greater than 300 psig and a temperature greater than the condensation point of butane at the second pressure; (f) condensing butane from the nitrogen by reducing the temperature of the nitrogen below the condensation point of butane at the second pressure while maintaining the nitrogen in a gaseous state; and (g) venting the nitrogen to the atmosphere.

In a second principal embodiment the invention provides a method of removing butane from a gaseous nitrogen headspace in a pressurized butane storage tank comprising: (a) releasing the gaseous nitrogen from the tank at a first pressure optionally greater than 30 psig; (b) compressing the nitrogen to a second pressure optionally greater than 300 psig and a temperature greater than the condensation point of butane at the second pressure; (c) condensing butane from the nitrogen by reducing the temperature of the nitrogen below the condensation point of butane at the second pressure while maintaining the nitrogen in a gaseous state; and (d) venting the nitrogen to the atmosphere.

A third principal embodiment relates to mobile systems capable of performing the methods of the current invention. Thus, in a third principal embodiment the invention provides a mobile system for removing butane from a gaseous nitrogen headspace in a pressurized butane storage tank comprising: (a) a mounting platform and a plurality of wheels rotatably secured thereto; (b) means for releasing the gaseous nitrogen at pressures optionally greater than 30 psig from the pressurized butane storage tank, wherein the gaseous nitrogen comprises a partial pressure of gaseous butane; (c) a compressor in fluid communication with the means for releasing, capable of increasing the pressure of the gaseous nitrogen to optionally greater than 300 psig and a temperature greater than the condensation point of butane at the second pressure; (d) a condenser in fluid communication with the compressor, capable of condensing butane from the nitrogen by reducing the temperature of the nitrogen below the condensation point of butane while maintaining the nitrogen in a gaseous state; (c) a high-pressure liquid separator capable of separating the condensed butane from the nitrogen while maintaining the condensed butane in a liquid state; and (f) means for venting the nitrogen to the atmosphere.

### Discussion of Subembodiments

In any of the principal embodiments, the liquid butane, after condensation from the nitrogen, is optionally returned to the original butane tank, and is preferably used for blending into a tank or stream of finished gasoline or diluent at a tank farm. Exemplary blending processes are described, for example, in U.S. Pat. Nos. 6,679,302, 7,631,671, 8,192,510, and 9,637,685.

The butane storage tank can take any form in which butane is traditionally stored or transported and thus includes, for example, stationary tanks (a/k/a butane bullets), rail cars, tanker trucks, barge tanks, and shipping tanks (i.e. ship holds). The design of the tank is not critical although generally there must be at least one inlet port through which nitrogen is injected into the tank, and at least one outlet port through which the butane is expelled. The same valves can be used, or alternate valves can be installed, for adding butane to the tank or releasing the nitrogen from the tank.

The nitrogen released from the tank will be contaminated predominantly by butane although other VOCs can be present depending on the content of the liquid butane and the temperature of the tank. In various subembodiments the liquid butane and/or gaseous butane mixed in the nitrogen will comprise greater than 50%, 65%, 80%, 90%, 95%, or 98% n-butane and isobutane. In further subembodiments the liquid butane and or gaseous butane mixed in the nitrogen comprises greater than 90%, 95%, 98%, or 99% n-butane, isobutane, propane, and ethane.

The tank itself can be defined based on various parameters including its pressure. Thus, in some embodiments the tank has a pressure greater than 15 psig, 30 psig, 60 psig, or 90 psig. In other embodiments the tank has a temperature greater than 70, 80, 90, or 100° F. The nitrogen gas, which is defined in this document to include any partial pressures contributed by VOCs, will typically occupy greater than 50%, 70%, or 90% of the tank volume.

The nitrogen gas inside the tank also can be characterized based on several different parameters, including the butane content in the nitrogen. Thus, in various embodiments, the partial pressure of the butane in the nitrogen gas ranges from about 1 to about 40 psi. Alternatively, one could state that the partial pressure of the butane is from 1% to 40% of the total pressure of the nitrogen gas.

The nitrogen gas can also be characterized based on the total content of VOCs, particularly C2-C6 alkanes. Thus, in one embodiment the nitrogen gas comprises a partial pressure of C2-C6 alkanes, and the C2-C6 alkanes comprise greater than 90% butane based on the partial pressures of the C2-C6 alkanes and the butane.

In operation, the nitrogen will typically be released from the tank at a pressure greater than 30 psig. However, the nitrogen can also be released at lower or higher pressures, including pressures greater than 5, 15, 25, 45, 60 or 90 psig.

After releasing the nitrogen from the tank, and compressing it, the butane will typically be condensed while maintaining the second pressure of the nitrogen substantially constant. I.e., there will be no loss in pressure other than loss of partial pressure from the butane, which by definition does not constitute a “substantial” difference.

Propane can also be condensed from the nitrogen and in, another embodiment, the invention provides condensing propane from the nitrogen by reducing the temperature of the nitrogen below the condensation point of propane while maintaining the nitrogen in a gaseous state. It will be understood by workers skilled in the art that the propane and butane can be condensed from the nitrogen in the same or different steps. When different steps are employed, it will be further understood that the propane and butane can be condensed with or without pressurizing the nitrogen between the steps, and that the propane and butane can be condensed from the nitrogen at substantially the same or different pressures.

In another embodiment, the method further comprises condensing ethane from the nitrogen by reducing the temperature of the nitrogen below the condensation point of ethane while maintaining the nitrogen in a gaseous state. Once again, the ethane and propane can be condensed from the nitrogen in the same or different steps. When different steps are employed, it will be further understood that the ethane and propane can be condensed from the nitrogen in different steps without pressurizing the nitrogen between the steps, and that the ethane and propane can be condensed from the nitrogen at substantially the same or different pressures.

### DESCRIPTION OF THE FIGURES

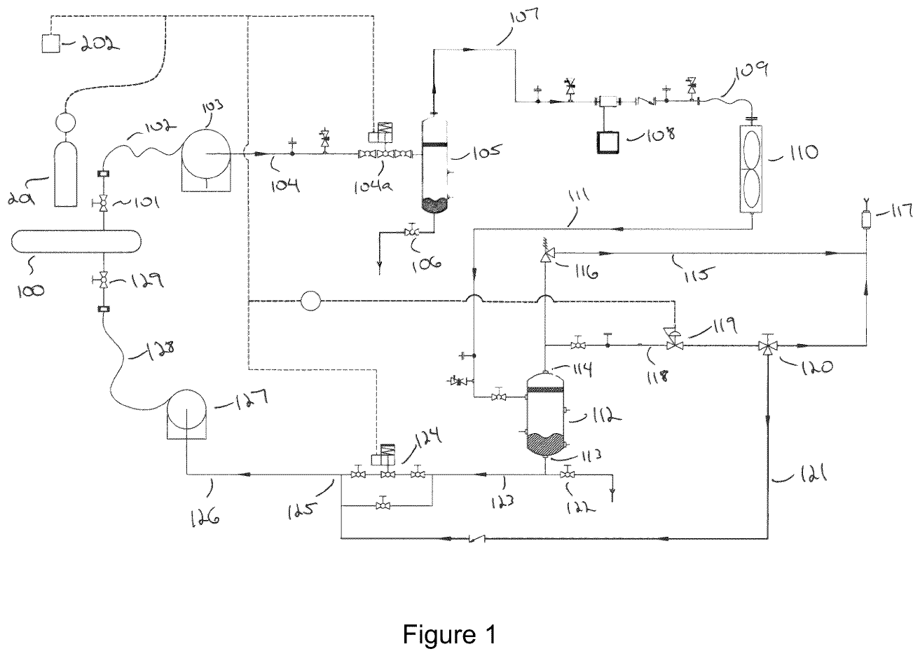

In FIG. 1 there is depicted a schematic of and exemplary butane removal system for carrying out the processes of the current invention based on compression and condensation of the VOCs in the gaseous headspace of a butane tank pressurized by gaseous nitrogen. The debutanization process depicted in FIG. 1 is based on a nitrogen stream principally contaminated by butane and relies on temperature reduction in the nitrogen stream while maintaining a substantially constant pressure to condense the butane and any lighter hydrocarbons such as propane and ethane. Solid lines with arrows depict fluid flow through the system, whereas dashed lines depict various pneumatic and/or digital controls.

Butane tank 100 is a stationary butane bullet of the type commonly seen at petroleum tank farms, used for blending butane into gasoline and other petroleum products to optimize the vapor pressure, viscosity, or other physical properties of the petroleum products, as described more thoroughly in, for example, U.S. Pat. Nos. 6,799,302, 7,631,671, and 9,321,977, the contents of which being hereby incorporated by reference. It will be recognized, however, that the tank need not be stationary, and that other tanks used for storing or transporting butane can also be used in the methods of the current invention, including railway cars and tanker truck cars.

Any tank used for transporting and/or storing butane that is pumped out by means of nitrogen pressurization is suitable for practicing the methods of the current invention. The tank will commonly exceed 5,000, 20,000, or even 50,000 gallons in volume. The pressure inside the tank when nitrogen is released from the tank can exceed 5 psig, 10 psig, 15 psig, 30 psig, 50 psig, 70 psig, or even 90 psig, and will commonly range from about 15 to about 200 psig, or from about 30 to about 100 psig.

In operation, tank 100 will be substantially empty of liquids and substantially full of highly pressurized nitrogen gas comprising butane and other VOCs commonly found in commercial sources of butane, usually C2-C6 alkanes such as ethane, propane, pentane, and hexane, most commonly ethane and propane. When the pressure of nitrogen is given in this document, it will be understood that the pressure in not a partial pressure but includes all components including butane and other VOCs. The pressure of the nitrogen gas, including the VOCs, can exceed 5 psig, 10 psig, 15 psig, 30 psig, 50 psig, 70 psig, or even 90 psig, and will commonly range from about 15 to about 200 psig, or from about 30 to about 100 psig.

The pressure of the butane in the nitrogen gas will commonly make up from 1% to 40%, 2% to 20%, or 3% to 10% of the total pressure of the gas in the tank, depending primarily on the temperature of the tank. The temperature of the tank can exceed 80, 90, or even 100 degrees Fahrenheit. The butane will typically be part of a mixture of VOCs selected from C2-C6 alkanes predominantly comprising butane. For example, the mixture of VOCs will typically comprise greater than 90%, 95%, or 98% butane based on the partial pressures of the VOCs and the butane.

Tank 100 comprises an outlet port and valve 101 in fluid communication with the gaseous headspace that feeds eduction line 102, shown as a flexible hose stored in hose reel 103 in FIG. 1. Valve 101, referred to elsewhere in this document as the “means for releasing,” will control the rate and pressure at which nitrogen flows through eduction line 102. Hoses and hose reels are employed on this particular system because the system is mounted on a portable trailer for servicing butane tanks in multiple locations. The hoses are simply disconnected from the butane tank, reeled into the hose reels, and any controls severed, after the unit has completed its evacuation of nitrogen from the tank.

A stationary pipe 104 conveys the nitrogen from hose reel 103 to an inlet liquid trap 105, with flow under the controlled coordination of a solenoid operated valve and valve system 104a. Inlet liquid trap 105 separates any liquids mixed in the gaseous nitrogen stream from the gaseous nitrogen prior to compression of the gaseous stream. Valve 106 controls the discharge of liquids from inlet liquid trap 105.

From liquid trap 105, nitrogen flows through line 107 to a variable frequency compressor (VFD) 108 designed to compress the nitrogen to a pressure of 500 psig and a temperature greater than 290° F. to maintain the butane in a gaseous state. Temperature and pressure transducers are included to monitor the temperature and pressure of the nitrogen before and after the compression stage, and to modulate the temperature and pressure when necessary.

After compression, the butane is transmitted through line 109 to a condenser unit 110, where the nitrogen is cooled to below the condensation temperature of butane at 500 psi (i.e. ˜240° F.). Transmission line 111 then carries the gaseous nitrogen and condensed liquid butane to a high pressure liquid trap 112 which releases the liquid butane through outlet 113 at the bottom of the unit and releases nitrogen through outlet 114 at the top of the unit.

Thereafter nitrogen is either released from the system through line 115, which travels through a pressure relief valve 116 set at, e.g., from 100 to 400 psig, and eventual discharge through air vent 117 (referred to elsewhere as the “means for venting”) at a maximum discharge pressure for the system, typically no more than 50, 35, 20, or 10 psig. Additional nitrogen exiting the liquid trap is directed through line 118, where it is optionally monitored for liquids and potentially returned to butane tank 100 through the controlled interaction of pressure regulator 119 and three-way valve 120, through nitrogen return line 121.

In like manner, any butane leaving the bottom of liquid trap 112 is either discharged through discharge valve 122 or returned to butane tank 100 through butane return line 123 and the controlled coordination of a solenoid operated valve 124 and three manual valves (unnumbered) (referred to elsewhere as the “means for returning”). Nitrogen return line 121 and butane return line 123 join at junction 125 and are subsequently transmitted via return line 126 through hose reel 127, through hose 128 and inlet valve 129 pack into butane tank 100.

In the system depicted in FIG. 1, the pneumatic and/or digital control will originate from nitrogen tank 201 and/or central processing unit (CPU) 202. Pressure and temperature transducers:

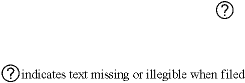

are preferably located throughout the system to facilitate control and oversight.

### OTHER EMBODIMENTS

Embodiment 1) A method of delivering butane comprising:
providing a pressurized butane storage tank comprising a liquid consisting essentially of butane and a gaseous headspace, wherein the tank comprises a liquid inlet port and a liquid outlet port in fluid communication with the liquid and a gas inlet port and a gas outlet port in fluid communication with the gaseous headspace;
injecting nitrogen gas into the tank through the gas inlet port at a pressure sufficient to expel liquid butane through the liquid outlet port;
retaining the gaseous nitrogen in the tank in the headspace for a time and at a temperature sufficient to form a gaseous mixture of butane and nitrogen;
releasing the gaseous nitrogen from the tank at a first pressure optionally greater than 30 psig;
compressing the nitrogen to a second pressure optionally greater than 300 psig and a temperature greater than the condensation point of butane at the second pressure;
condensing butane from the nitrogen by reducing the temperature of the nitrogen below the condensation point of butane at the second pressure while maintaining the nitrogen in a gaseous state; and
venting the nitrogen to the atmosphere.

Embodiment 2) The method of embodiment 1, further comprising blending the expelled liquid butane into a tank or stream of finished gasoline or diluent.

Embodiment 3) The method of embodiment 1 or 2, wherein the butane storage tank is a stationary tank, a rail car, a tanker truck, or a barge tank or shipping tank.

Embodiment 4) The method of any of embodiments 1-3, wherein the gas inlet port and the liquid inlet port are the same or different.

Embodiment 5) The method of any of embodiments 1-3, wherein the gas inlet port and the liquid outlet port are the same or different.

Embodiment 6) The method of any of embodiments 1-3, wherein the gas outlet port and the liquid outlet port are the same or different.

Embodiment 7) The method of any of embodiments 1-3, wherein the gas outlet port and the liquid inlet port are the same or different.

Embodiment 8) The method of any of embodiments 1-7, further comprising transmitting the condensed butane to a fuel storage tank which is the same as or different than the butane storage tank.

Embodiment 9) The method of 1-8, wherein the butane is condensed while maintaining the second pressure of the nitrogen substantially constant.

Embodiment 10) The method of any of embodiments 1-9, further comprising condensing propane from the nitrogen by reducing the temperature of the nitrogen below the condensation point of propane while maintaining the nitrogen in a gaseous state.

Embodiment 11) The method of any of embodiments 1-10, wherein the propane and butane are condensed from the nitrogen in the same step.

Embodiment 12) The method of any of embodiments 1-10, wherein the propane and butane are condensed from the nitrogen in different steps.

Embodiment 13) The method of any of embodiments 1-10, wherein the propane and butane are condensed from the nitrogen in different steps without pressurizing the nitrogen between the steps.

Embodiment 14) The method of any of embodiments 1-10, wherein the propane and butane are condensed from the nitrogen at substantially the same pressures.

Embodiment 15) The method of any of embodiments 1-14, further comprising condensing ethane from the nitrogen by reducing the temperature of the nitrogen below the condensation point of ethane while maintaining the nitrogen in a gaseous state.

Embodiment 16) The method of any of embodiments 1-15, wherein the ethane and propane are condensed from the nitrogen in the same step.

Embodiment 17) The method of any of embodiments 1-15, wherein the ethane and propane are condensed from the nitrogen in different steps.

Embodiment 18) The method of 1-15, wherein the ethane and propane are condensed from the nitrogen in different steps without pressurizing the nitrogen between the steps.

Embodiment 19) The method of any of embodiments 1-15, wherein the ethane and propane are condensed from the nitrogen at substantially the same pressures.

Embodiment 20) The method of any of embodiments 1-19, wherein the liquid butane comprises greater than 50%, 65%, 80%, 90%, 95%, or 98% n-butane and isobutane.

Embodiment 21) The method of any of embodiments 1-20, wherein the liquid butane comprises greater than 90%, 95%, 98%, or 99% n-butane, isobutane, propane, and ethane.

Embodiment 22) The method of any of embodiments 1-19, wherein the liquid butane comprises:
greater than 50%, 65%, 80%, 90%, 95%, or 98% n-butane and isobutane; and
ii) greater than 90%, 95%, 98%, or 99% n-butane, isobutane, propane, and ethane.

Embodiment 23) The method of any of embodiments 1-22, wherein the pressurized butane tank has a pressure greater than 5 psig, 10 psig, 15 psig, 30 psig, 60 psig, or 90 psig.

Embodiment 24) The method of any of embodiments 1-23, wherein the pressurized butane tank has a temperature greater than 70, 80, 90, or 100 F.

Embodiment 25) The method of any of embodiments 1-24, wherein the first pressure is greater than 30 or 60 or 90 psig.

Embodiment 26) The method of any of embodiments 1-25, wherein the partial pressure of the butane is from 1% to 40% of the total pressure of the nitrogen gas.

Embodiment 27) The method of any of embodiments 1-26, wherein the nitrogen gas comprises a partial pressure of C2-C6 alkanes, and the C2-C6 alkanes comprise greater than 90% butane based on the partial pressures of the C2-C6 alkanes and the butane.

Embodiment 28) The method of any of embodiments 1-27, wherein the nitrogen gas occupies greater than 50%, 70%, or 90% of the tank volume.

Embodiment 29) A method of removing butane from a gaseous nitrogen headspace in a pressurized butane storage tank comprising:
releasing the gaseous nitrogen from the tank at a first pressure optionally greater than 30 psig;
compressing the nitrogen to a second pressure optionally greater than 300 psig and a temperature greater than the condensation point of butane at the second pressure;
condensing butane from the nitrogen by reducing the temperature of the nitrogen below the condensation point of butane at the second pressure while maintaining the nitrogen in a gaseous state; and
venting the nitrogen to the atmosphere.

Embodiment 30) The method of embodiment 29, wherein the butane storage tank is a stationary tank, a rail car, a tanker truck, or a barge tank or shipping tank.

Embodiment 31) A mobile system for removing butane from a gaseous nitrogen headspace in a pressurized butane storage tank comprising:
a mounting platform and a plurality of wheels rotatably secured thereto;
means for releasing the gaseous nitrogen at a first pressure optionally greater than 30 psig from the pressurized butane storage tank, wherein the gaseous nitrogen comprises a partial pressure of gaseous butane;
a compressor in fluid communication with the means for releasing, capable of increasing the pressure of the gaseous nitrogen to a second pressure optionally greater than 300 psig and a temperature greater than the condensation point of butane at the second pressure;
a condenser in fluid communication with the compressor, capable of condensing butane from the nitrogen by reducing the temperature of the nitrogen below the condensation point of butane while maintaining the nitrogen in a gaseous state;
a high-pressure liquid separator capable of separating the condensed butane from the nitrogen while maintaining the condensed butane in a liquid state; and
means for venting the nitrogen to the atmosphere.

Embodiment 32) The system of embodiment 31, further comprising means for returning the condensed butane to the liquid butane tank.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of delivering butane comprising:
a) providing a pressurized butane storage tank comprising a liquid consisting essentially of butane and a gaseous headspace, wherein the tank comprises a liquid inlet port and a liquid outlet port in fluid communication with the liquid and a gas inlet port and a gas outlet port in fluid communication with the gaseous headspace;
b) injecting nitrogen gas into the tank through the gas inlet port at a pressure sufficient to expel liquid butane through the liquid outlet port;
c) retaining the gaseous nitrogen in the tank in the headspace for a time and at a temperature sufficient to form a gaseous mixture of butane and nitrogen;
d) releasing the gaseous nitrogen from the tank at a first pressure optionally greater than 30 psig;
e) compressing the nitrogen to a second pressure optionally greater than 300 psig and a temperature greater than the condensation point of butane at the second pressure;
f) condensing butane from the nitrogen by reducing the temperature of the nitrogen below the condensation point of butane at the second pressure while maintaining the nitrogen in a gaseous state; and
g) venting the nitrogen to the atmosphere.

2. The method of claim 1, further comprising blending the expelled liquid butane into a tank or stream of finished gasoline or diluent.

3. The method of claim 1, wherein the butane storage tank is a stationary tank, a rail car, a tanker truck, or a barge tank or shipping tank.

4. The method of claim 1, wherein the gas inlet port and the liquid inlet port are the same or different.

5. The method of claim 1, wherein the gas inlet port and the liquid outlet port are the same or different.

6. The method of claim 1, wherein the gas outlet port and the liquid outlet port are the same or different.

7. The method of claim 1, wherein the gas outlet port and the liquid inlet port are the same or different.

8. The method of claim 1, further comprising transmitting the condensed butane to a fuel storage tank which is the same as or different than the butane storage tank.

9. The method of claim 1, wherein the butane is condensed while maintaining the second pressure of the nitrogen substantially constant.

10. The method of claim 1, further comprising condensing propane from the nitrogen by reducing the temperature of the nitrogen below the condensation point of propane while maintaining the nitrogen in a gaseous state.

11. The method of claim 10, wherein the propane and butane are condensed from the nitrogen in the same step.

12. The method of claim 10, wherein the propane and butane are condensed from the nitrogen in different steps.

13. The method of claim 10, wherein the propane and butane are condensed from the nitrogen in different steps without pressurizing the nitrogen between the steps.

14. The method of claim 10, wherein the propane and butane are condensed from the nitrogen at substantially the same pressures.

15. The method of claim 10, further comprising condensing ethane from the nitrogen by reducing the temperature of the nitrogen below the condensation point of ethane while maintaining the nitrogen in a gaseous state.

16. The method of claim 15, wherein the ethane and propane are condensed from the nitrogen in the same step.

17. The method of claim 15, wherein the ethane and propane are condensed from the nitrogen in different steps.

18. The method of claim 15, wherein the ethane and propane are condensed from the nitrogen in different steps without pressurizing the nitrogen between the steps.

19. The method of claim 15, wherein the ethane and propane are condensed from the nitrogen at substantially the same pressures.

20. The method of claim 1, wherein the liquid butane comprises greater than 50%, 65%, 80%, 90%, 95%, or 98% n-butane and isobutane.

21. The method of claim 1, wherein the liquid butane comprises greater than 90%, 95%, 98%, or 99% n-butane, isobutane, propane, and ethane.

22. The method of claim 1, wherein the liquid butane comprises:
i) greater than 50%, 65%, 80%, 90%, 95%, or 98% n-butane and isobutane; and
ii) greater than 90%, 95%, 98%, or 99% n-butane, isobutane, propane, and ethane.

23. The method of claim 1, wherein the pressurized butane tank has a pressure greater than 5 psig, 10 psig, 15 psig, 30 psig, 60 psig, or 90 psig.

24. The method of claim 1, wherein the pressurized butane tank has a temperature greater than 70, 80, 90, or 100 F.

25. The method of claim 1, wherein the first pressure is greater than 30 or 60 or 90 psig.

26. The method of claim 1, wherein the partial pressure of the butane is from 1% to 40% of the total pressure of the nitrogen gas.

27. The method of claim 1, wherein the nitrogen gas comprises a partial pressure of C2-C6 alkanes, and the C2-C6 alkanes comprise greater than 90% butane based on the partial pressures of the C2-C6 alkanes and the butane.

28. The method of claim 1, wherein the nitrogen gas occupies greater than 50%, 70%, or 90% of the tank volume.

29. A method of removing butane from a gaseous nitrogen headspace in a pressurized butane storage tank comprising:
a) releasing the gaseous nitrogen from the tank at a first pressure optionally greater than 30 psig;
b) compressing the nitrogen to a second pressure optionally greater than 300 psig and a temperature greater than the condensation point of butane at the second pressure;
c) condensing butane from the nitrogen by reducing the temperature of the nitrogen below the condensation point of butane at the second pressure while maintaining the nitrogen in a gaseous state; and
d) venting the nitrogen to the atmosphere.

30. The method of claim 29, wherein the butane storage tank is a stationary tank, a rail car, a tanker truck, or a barge tank or shipping tank.

31. A mobile system for removing butane from a gaseous nitrogen headspace in a pressurized butane storage tank comprising:
a) a mounting platform and a plurality of wheels rotatably secured thereto;
b) means for releasing the gaseous nitrogen at a first pressure optionally greater than 30 psig from the pressurized butane storage tank, wherein the gaseous nitrogen comprises a partial pressure of gaseous butane;
c) a compressor in fluid communication with the means for releasing, capable of increasing the pressure of the gaseous nitrogen to a second pressure optionally greater than 300 psig and a temperature greater than the condensation point of butane at the second pressure;
d) a condenser in fluid communication with the compressor, capable of condensing butane from the nitrogen by reducing the temperature of the nitrogen below the condensation point of butane while maintaining the nitrogen in a gaseous state;
e) a high-pressure liquid separator capable of separating the condensed butane from the nitrogen while maintaining the condensed butane in a liquid state; and
f) means for venting the nitrogen to the atmosphere.

32. The system of claim 31, further comprising means for returning the condensed butane to the liquid butane tank.
